(19) United States
(12) Patent Application Publication
(10) Pub. No.: US 2016/0345841 A1
(43) Pub. Date: Dec. 1, 2016

(54) METHOD AND APPARATUS FOR ESTIMATING PHYSIOLOGICAL INDEX OF USER AT MAXIMAL EXERCISE LEVEL BASED ON RATING OF PERCEIVED EXERTION

(71) Applicant: SAMSUNG ELECTRONICS CO., LTD., Suwon-si (KR)

(72) Inventors: Dae-Geun JANG, Yongin-si (KR); Byunghoon KO, Hwaseong-si (KR); SangKon BAE, Seongnam-si (KR)

(73) Assignee: SAMSUNG ELECTRONICS CO., LTD., Suwon-si (KR)

(21) Appl. No.: 14/984,682

(22) Filed: Dec. 30, 2015

(30) Foreign Application Priority Data
May 27, 2015 (KR) 10-2015-0073733

(51) Int. Cl. A61B 5/0205; A63B 24/00; A61B 5/08; A61B 5/1455; A61B 5/11

(52) U.S. Cl. CPC A61B 5/0205; A61B 5/14552; A61B 5/1118; A61B 5/0816; A63B 24/0062

(57) ABSTRACT

A method of estimating a physiological index of a user includes measuring a rating of perceived exertion (RPE) and a first physiological index of a user at different exercise levels of an exercise or a daily activity having a varying exercise intensity, and estimating a second physiological index of the user at a maximal exercise level based on the RPE and the first physiological index.

### CROSS-REFERENCE TO RELATED APPLICATION(S)

This application claims the benefit under 35 USC 119(a) of Korean Patent Application No. 10-2015-0073733 filed on May 27, 2015, in the Korean Intellectual Property Office, the entire disclosure of which is incorporated herein by reference for all purposes.

### BACKGROUND

1. Field

The following description relates to a method and an apparatus for estimating a physiological index of a user at a maximal exercise level corresponding to a physical fitness or an exercise capability of the user based on a rating of perceived exertion (RPE).

2. Description of Related Art

An incremental maximal or submaximal exercise test is generally used to evaluate cardiopulmonary fitness. However, in a case of a risk group including children, elderly people, and adults having a potential risk of developing, for example, cardiovascular and respiratory diseases, such an incremental maximal or submaximal exercise test may not be readily conducted due to a physical stress or an injury that may occur during exercise. In addition, when such tests are conducted on a physically inactive test subject, accuracy and reliability of test results may be degraded due to, for example, local muscular fatigue such as a fatigue in quadriceps femoris muscles.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method of estimating a physiological index of a user includes measuring a rating of perceived exertion (RPE) and a first physiological index of a user at different exercise levels of an exercise or a daily activity having a varying exercise intensity; and estimating a second physiological index of the user at a maximal exercise level based on the RPE and the first physiological index.

The estimating of the second physiological index may include deriving a personalized regression equation for the user based on a relationship between the RPE and the first physiological index; and estimating the second physiological index at the maximal exercise level using the personalized regression equation.

The deriving of the personalized regression equation may include generating a graph based on the relationship between the RPE and the first physiological index; and deriving the personalized regression equation from the graph.

The generating of the graph may include generating the graph by approximating a value of the first physiological index corresponding to the RPE.

The estimating of the second physiological index may include estimating the second physiological index at the maximal exercise level by substituting, in the personalized regression equation, an RPE corresponding to the maximal exercise level.

The RPE and the first physiological index may be simultaneously measured at the different exercise levels.

The RPE may be expressed by a Borg scale, an OMNI scale, a Likert scale, or a visual analog scale of perceived exertion.

The first physiological index may include any one or more of a heart rate, a pulse rate, a respiratory rate, a blood pressure, a stroke volume, a cardiac output, a ventilation (VE), an oxygen uptake (VO₂), an oxygen concentration in exhaled air (FeO₂), a carbon dioxide concentration in exhaled air (FeCO₂), a ventilatory equivalent for oxygen (EqO₂), a respiratory exchange ratio (RER), a metabolic equivalent of task (MET), a blood lactate level, and a blood oxygen saturation (SpO₂) level.

The different exercise levels may include at least two exercise levels selected from exercise levels corresponding to a warming-up stage prior to an exercise, an exercising stage during the exercise, and a cooling-down stage subsequent to the exercise.

The method may further include evaluating an exercise capability of the user based on the estimated second physiological index.

The method may further include generating an exercise program suitable for the exercise capability of the user based on a result of the evaluating; and adjusting an exercise level and intensity for the user based on the exercise program.

The method may further include comparing the exercise capability of the user to a preset standard exercise capability for a gender and an age of the user; and providing a result of the comparing as feedback to the user.

The method may further include calculating a metabolic syndrome risk of the user based on the estimated second physiological index.

In another general aspect, a non-transitory computer-readable storage medium stores instructions to control computing hardware to perform the method described above.

In another general aspect, an apparatus for estimating a physiological index of a user includes a measurer configured to measure a rating of perceived exertion (RPE) and a first physiological index of a user at different exercise levels of an exercise having a varying exercise intensity; and a processor configured to estimate a second physiological index of the user at a maximal exercise level based on the RPE and the first physiological index.

The processor may be further configured to derive a personalized regression equation for the user based on a relationship between the RPE and the first physiological index, and estimate the second physiological index at the maximal exercise level using the personalized regression equation.

The processor may be further configured to generate a graph by approximating a value of the first physiological index corresponding to the RPE, and derive the personalized regression equation from the graph.

The processor may be further configured to estimate the second physiological index at the maximal exercise level by substituting, in the personalized regression equation, an RPE corresponding to the maximal exercise level.

The processor may be further configured to evaluate an exercise capability of the user based on the estimated second physiological index, generate an exercise program suitable for the exercise capability of the user, and adjust an exercise level and intensity for the user based on the exercise program.

The processor may be further configured to evaluate the exercise capability of the user based on the estimated second physiological index, compare the exercise capability of the user to a preset standard exercise capability for a gender and an age of the user, and provide a result of the comparing as feedback to the user.

Other features and aspects will be apparent from the following detailed description, the drawings, and the claims.

### BRIEF DESCRIPTION OF THE DRAWINGS

FIG. 1 is a flowchart illustrating an example of a method of estimating a physiological index of a user.

FIG. 2 is a flowchart illustrating another example of a method of estimating a physiological index of a user.

FIG. 3 is a diagram illustrating an example of a method of measuring a first physiological index and a rating of perceived exertion (RPE) during an exercise having a varying exercise level.

FIGS. 4A and 4B illustrate examples of a method of generating a graph based on a relationship between an RPE and a first physiological index.

FIGS. 5A and 5B illustrate examples of a method of estimating a second physiological index of a user at a maximal exercise level based on an RPE and a first physiological index.

FIG. 6 is a flowchart illustrating another example of a method of estimating a physiological index of a user.

FIG. 7 is a flowchart illustrating another example of a method of estimating a physiological index of a user.

FIG. 8 is a diagram illustrating an example of an apparatus for estimating a physiological index of a user.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The terminology used herein is for the purpose of describing particular examples only, and is not intended to limit the disclosure. As used herein, the singular forms “a,” “an,” and “the” are intended to include the plural forms as well, unless the context clearly indicates otherwise. As used herein, the terms “comprises,” “comprising,” “includes,” “including,” and “have” specify the presence of stated features, numbers, operations, elements, components, and combinations thereof, but do not preclude the presence or addition of one or more other features, numbers, operations, elements, components, and combinations thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

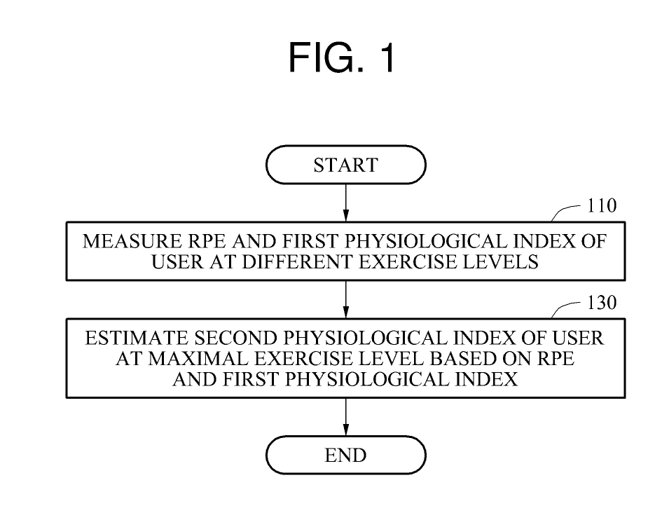

FIG. 1 is a flowchart illustrating an example of a method of estimating a physiological index of a user.

Referring to FIG. 1, in operation 110, an apparatus for estimating a physiological index of a user, hereinafter referred to as a physiological index estimating apparatus, measures a rating of perceived exertion (RPE) and a first physiological index of a user.

For example, the physiological index estimating apparatus may measure an RPE and a first physiological index of a user during an exercise having a varying exercise level or intensity, for example, an exercise using equipment, for example, running on a treadmill, riding on a bicycle ergometer, and stepping on a bench step, and a general exercise, for example, jogging, walking, swimming, mountain climbing, and stair climbing.

The physiological index estimating apparatus measures the RPE and the first physiological index at different exercise levels during an exercise. The different exercise levels are at least two exercise levels selected from exercise levels corresponding to, for example, a warming-up stage prior to an exercise, a low-level exercising stage, a moderate-level exercising stage, and a high-level exercising stage during the exercise, and a cooling-down stage subsequent to the exercise.

In one example, the physiological index estimating apparatus measures an RPE and a first physiological index at exercise levels other than a maximal exercise level to enable users including children, elderly people, and adults included in a risk group for health reasons to perform an exercise, for example, a low-level and a moderate-level exercise, corresponding to an exercise level of a daily activity to prevent a physical stress and an injury that may occur due to excessive exercising. The maximal exercise level may include, for example, a maximal exercise intensity corresponding to the original Borg scale of 19 to 20 for perceived exertion, and also a submaximal exercise intensity slightly lower than the maximal exercise intensity which corresponds to the Borg scale of 16 to 18.

For example, the physiological index estimating apparatus may measure an RPE and a first physiological index at each of a first exercise level corresponding to the warming-up stage, and a fourth and a sixth exercise level corresponding to the moderate-level exercising stage. The RPE and the first physiological index at each exercise level may be simultaneously measured.

The physiological index estimating apparatus estimates a second physiological index of the user at the maximal exercise level based on the RPE and the first physiological index measured at specific time points during the exercise, and thus need not continuously monitor a change in the RPE and the first physiological index during the exercise.

The physiological index estimating apparatus may be a wearable device including sensors in various forms, for example, a watch type, a bracelet type, a chest type, a patch type, and an in-ear type, or a mobile device connected to a wearable device through wired or wireless communication.

The physiological index estimating apparatus measures the first physiological index using various sensors. The first physiological index is a physiological index indicating a metabolic characteristic of the user and may be, for example, a heart rate (HR), a pulse rate, a respiratory rate, a blood pressure, a stroke volume, a cardiac output, a ventilation (VE), an oxygen uptake (VO₂), an oxygen concentration in exhaled air (FeO₂), a carbon dioxide concentration in exhaled air (FeCO₂), a ventilatory equivalent for oxygen (EqO₂), a respiratory exchange ratio (RER), a metabolic equivalent of task (MET), or a blood oxygen saturation (SpO₂) level. The physiological index estimating apparatus may measure a single first physiological index or a plurality of first physiological indices of a user.

The RPE indicates a physical exertion level that is subjectively perceived or recognized by a user performing an exercise. The RPE may be affected by, for example, a physical fitness of the user, environmental conditions, and a general fatigue level.

The RPE may be expressed by the Borg scale, the OMNI scale, the Likert scale, or the visual analog scale of perceived exertion, all of which are well known to one of ordinary skill in the art. The Borg scale and the OMNI scale are shown in Table 1 and Table 2 below, respectively.

TABLE 1

| Borg's Category Scale | | Borg's Category-Ratio Scale | | |
|---|---|---|---|---|
| (Original) | | (Revised) | | |
| Scale | Description | Scale | Description | |
| 6 | | 0 | Nothing at all | |
| 7 | Very, very light | 0.3 | | |
| 8 | | 0.5 | Extremely weak | Just noticeable |
| 9 | Very light | 0.7 | | |
| 10 | | 1 | Very weak | |
| 11 | Fairly light | 1.5 | | |
| 12 | | 2 | Weak | Light |
| 13 | Somewhat hard | 2.5 | | |
| 14 | | 3 | Moderate | |
| 15 | Hard | 4 | | |
| 16 | | 5 | Strong | Heavy |
| 17 | Very hard | 6 | | |
| 18 | | 7 | Very strong | |
| 19 | Very, very hard | 8 | | |
| 20 | | 9 | | |
| | | 10 | Extremely strong | Maximal |
| | | 11 | | |

TABLE 2

| | |
|---|---|
| for Adults | for Children |

| | | | |
|---|---|---|---|
| 0 | Extremely easy | 0 | Not tired at all |
| 1 | | 1 | |
| 2 | Easy | 2 | Little tired |
| 3 | | 3 | |
| 4 | Somewhat easy | 4 | Getting more tired |
| 5 | | 5 | |
| 6 | Somewhat hard | 6 | Tired |
| 7 | | 7 | |
| 8 | Hard | 8 | Really tired |
| 9 | | 9 | |
| 10 | Extremely hard | 10 | Very, very tired |

Referring to Table 1, on the original Borg scale, an RPE corresponding to a maximal exercise level is expressed as level 19. On the revised Borg scale and the OMNI scale, an RPE corresponding to the maximal exercise level is expressed as level 10.

For example, when the physiological index estimating apparatus uses the OMNI scale illustrated in Table 2, the physiological index estimating apparatus inquires about an RPE of an exercise that is currently being performed by the user, for example, by asking a question “What is the exercise level of the exercise program?,” using a display or a speaker, and guides the user to select an answer from “extremely hard, hard, somewhat hard, somewhat easy, easy, and extremely easy,” or levels 0 through 10. The physiological index estimating apparatus measures the RPE of the user based on an input from the user or the selected answer, for example, the selected level, in accordance with the guidance.

In operation 130, the physiological index estimating apparatus estimates the second physiological index of the user at the maximal exercise level based on the RPE and the first physiological index. The physiological index estimating apparatus estimates the second physiological index of the user at the maximal exercise level by deriving a personalized regression equation for the user based on a relationship between the RPE and the first physiological index. The personalized regression equation may be a linear equation or a nonlinear equation depending on whether the relationship between the RPE and the first physiological index is linear or nonlinear.

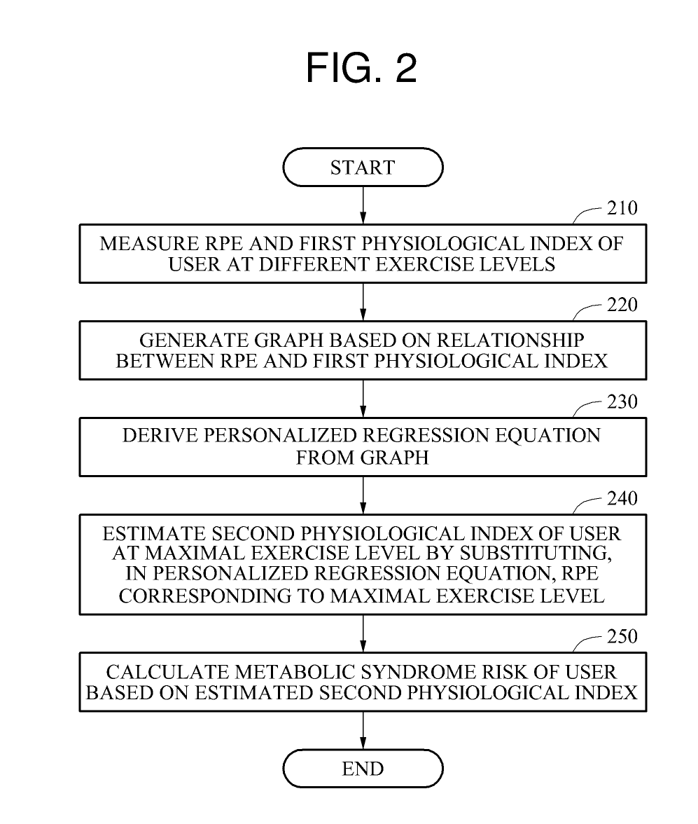

FIG. 2 is a flowchart illustrating another example of a method of estimating a physiological index of a user.

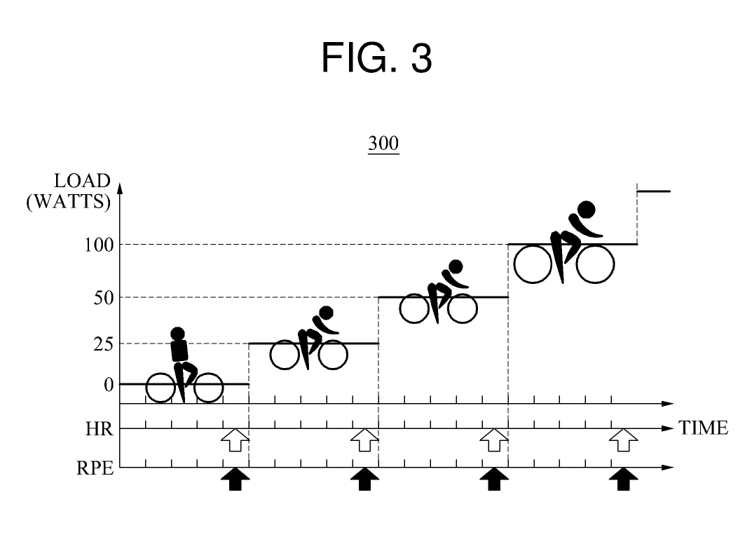

Referring to FIG. 2, in operation 210, a physiological index estimating apparatus measures an RPE and a first physiological index of a user at different exercise levels of an exercise having a varying exercise intensity. A method of measuring an RPE and a first physiological index of a user at different exercise levels will be described with reference to FIG. 3.

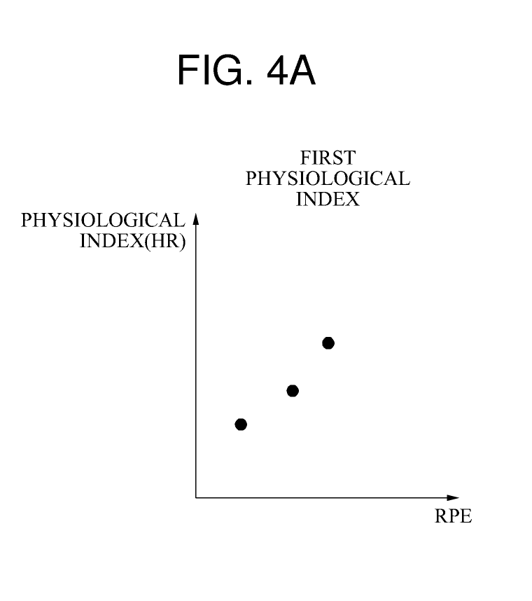

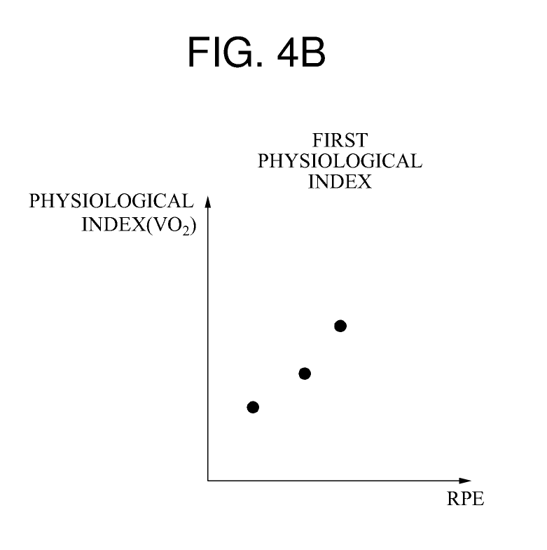

In operation 220, the physiological index estimating apparatus generates a graph based on a relationship between the RPE and the first physiological index. The physiological index estimating apparatus generates the graph by approximating a value of the first physiological index corresponding to the RPE. A method of generating such a graph will be described with reference to FIGS. 4A and 4B.

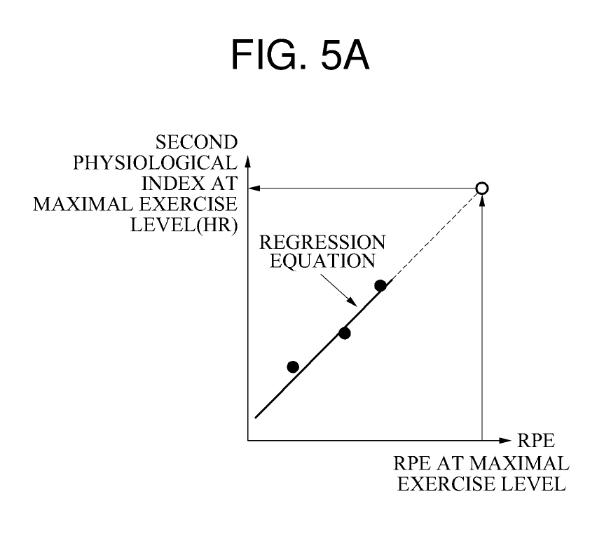

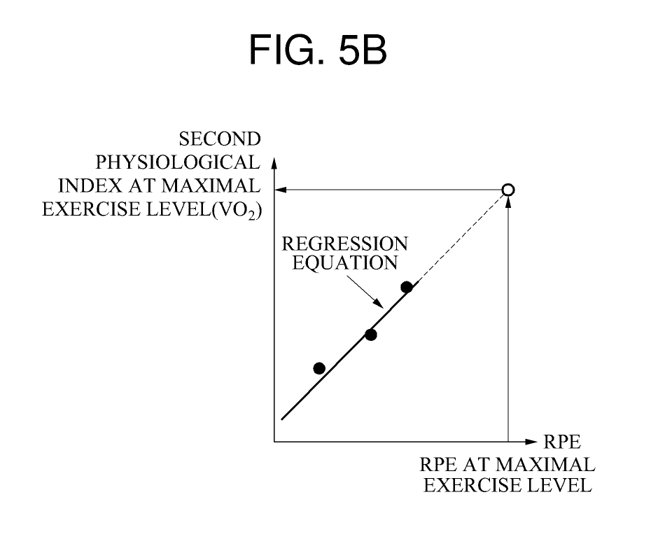

In operation 230, the physiological index estimating apparatus derives a personalized regression equation from the graph generated in operation 220. In operation 240, the physiological index estimating apparatus estimates a second physiological index at a maximal exercise level by substituting, in the personalized regression equation derived in operation 230, an RPE corresponding to the maximal exercise level. A method of estimating a second physiological index at a maximal exercise level will be described with reference to FIGS. 5A and 5B.

In operation 250, the physiological index estimating apparatus calculates a metabolic syndrome risk of the user based on the estimated second physiological index.

FIG. 3 is a diagram illustrating an example of a method of measuring a first physiological index and an RPE during an exercise having a varying exercise level.

FIG. 3 illustrates a first physiological index, for example, an HR, and an RPE of a user measured when the user performs a cycling exercise.

An exercise having a varying exercise intensity may include a plurality of exercise levels corresponding to a warming-up stage prior to the exercise, a low-level, a moderate-level, and a high-level exercising stage, and a cooling-down stage subsequent to the exercise as illustrated in FIG. 3.

A physiological index estimating apparatus may measure, two or more times, an RPE and a first physiological index of a user at an exercise level among exercise levels corresponding to the stages excluding the high-level exercising stage. The RPE and the first physiological index may be simultaneously measured.

For example, the physiological index estimating apparatus may measure the RPE and the first physiological index in a steady state immediately before an exercise level changes during the exercise having a varying exercise intensity. In FIG. 3, arrow portions indicate a steady state at each exercise level at which the RPE and the first physiological index may be measured.

In one example, the steady state at each exercise level is a state in which an HR of a user remains constant in an exercise section corresponding to a constant exercise level. For example, when a change in a first physiological index, for example, an HR, of the user or an exercise duration at an exercise level satisfies a preset condition, the physiological index estimating apparatus may determine that the steady state at the exercise level has been attained and measure the first physiological index and the RPE.

The first physiological index in the steady state may be measured through sensors in various forms included in the physiological index estimating apparatus, and the RPE may be measured based on an RPE input from the user through an audio guidance or a display guidance.

For example, the physiological index estimating apparatus may measure, through the sensors, the first physiological index at the same time that the RPE is input from the user.

FIGS. 4A and 4B illustrate examples of a method of generating a graph based on a relationship between an RPE and a first physiological index.

FIGS. 4A and 4B illustrate graphs in which an RPE and a first physiological index measured three times at different exercise levels are indicated. In the graphs, an X axis indicates an RPE, and a Y axis indicates a first physiological index. FIG. 4A is a graph illustrating a relationship between an RPE and an HR, which is one first physiological index, and FIG. 4B is a graph illustrating a relationship between an RPE and a VO₂, which is another first physiological index. In the graphs, a value of the first physiological index corresponding to each RPE is indicated as a point.

A physiological index estimating apparatus generates a linear graph by approximating a value of a first physiological index corresponding to an RPE. For example, as illustrated in FIGS. 4A and 4B, the physiological index estimating apparatus generates a linear graph by connecting three points indicated in each graph. When the three points cannot be connected by a single straight line, the physiological index estimating apparatus generates the linear graph by approximating a value of the first physiological index corresponding to the RPE to a value closest to the straight line.

The physiological index estimating apparatus may generate a single linear graph by approximating points that are values of the first physiological index corresponding to each RPE to a position corresponding to a mean value of an angle between a plane and each line connecting the three points. The linear graph generated by the physiological index estimating apparatus may be different for each user.

The physiological index estimating apparatus derives, from the linear graph generated using the methods described above, a regression equation that most accurately represents the relationship between the RPE and the first physiological index. The derived regression equation may be a regression equation personalized for each user because a regression equation may be different for each user. The physiological index estimating apparatus may generate a linear graph using various methods other than the methods described above.

Also, a nonlinear relationship may be established between the RPE and the first physiological index measured at different exercise levels. In such a case, the physiological index estimating apparatus derives a nonlinear regression equation from a nonlinear graph.

FIGS. 5A and 5B illustrate examples of a method of estimating a second physiological index of a user at a maximal exercise level based on an RPE and a first physiological index.

FIGS. 5A and 5B illustrate graphs illustrating a personalized regression equation and a second physiological index of a user at a maximal exercise level that is estimated from the personalized regression equation. In the graphs, an X axis indicates an RPE, and a Y axis indicates a physiological index. FIG. 5A is a graph illustrating a relationship between an RPE and an HR, which is one first physiological index, and FIG. 5B is a graph illustrating a relationship between an RPE and a VO₂, which is another first physiological index.

A physiological index estimating apparatus estimates the second physiological index of the user at the maximal exercise level using the personalized regression equation derived from the graphs illustrated in FIGS. 4A and 4B. The second physiological index of the user at the maximal exercise level is an index indicating a physical fitness and an exercise capability of the user. As the second physiological index of the user at the maximal exercise level, a maximal HR (HRₘₐₓ), a maximal VO₂ (VO₂ₘₐₓ), a lactate threshold, a ventilatory threshold, or other maximal physiological index may be estimated.

The physiological index estimating apparatus estimates the second physiological index of the user at the maximal exercise level by substituting, in the personalized regression equation, an RPE corresponding to the maximal exercise level.

For example, referring to FIG. 5A, the personalized regression equation may be expressed as Y=10*X+2, wherein “Y” denotes a first physiological index, for example, an HR, and “X” denotes an RPE which is expressed on the original Borg scale. Based on the original Borg scale, an RPE corresponding to the maximal exercise level is level 19.

The physiological index estimating apparatus substitutes, in the regression equation, 19, which is the RPE corresponding to the maximal exercise level on the original Borg scale, as a value of X. The physiological index estimating apparatus obtains, Y, which is the first physiological index, as 192 from the regression equation, Y=10*19+2=192. The physiological index estimating apparatus then estimates 192 beats per minute (bpm) to be a second physiological index of the user at the maximal exercise level, for example, an HRₘₐₓ.

Similar to the method described with reference to FIG. 5A, the physiological index estimating apparatus also estimates a maximal oxygen uptake VO₂ₘₐₓ as the second physiological index of the user at the maximal exercise level as illustrated in FIG. 5B.

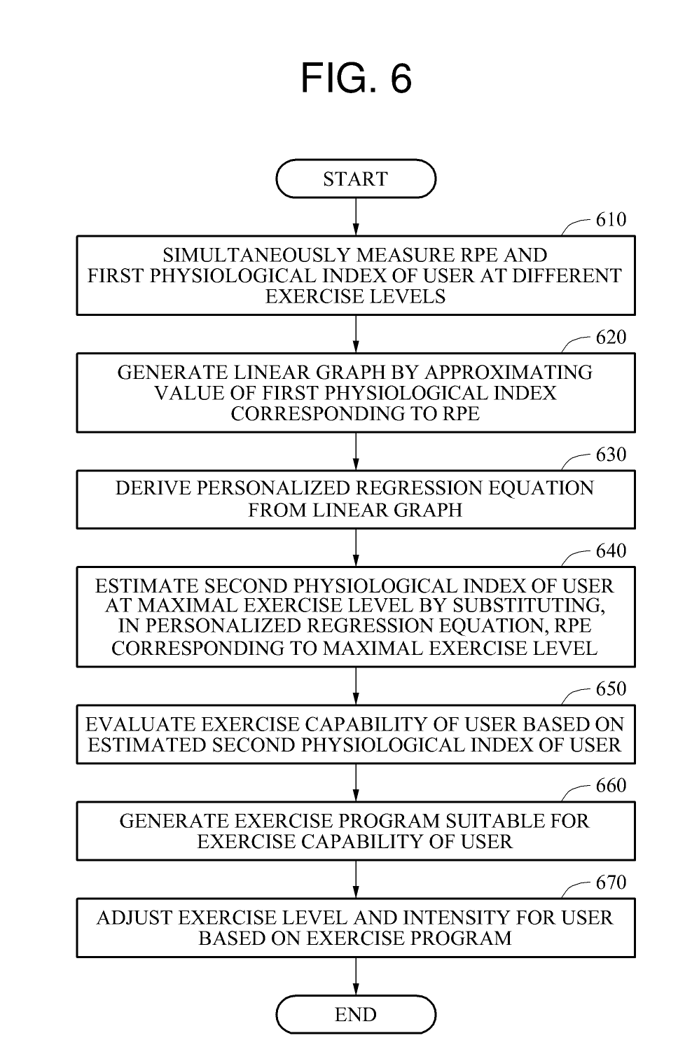

FIG. 6 is a flowchart illustrating another example of a method of estimating a physiological index of a user.

Referring to FIG. 6, in operation 610, a physiological index estimating apparatus measures an RPE and a first physiological index of a user at different exercise levels of an exercise having a varying exercise intensity. The physiological index estimating apparatus may measure a single first physiological index or a plurality of first physiological indices of the user.

In operation 620, the physiological index estimating apparatus generates a linear graph based on a relationship between the RPE and the first physiological index measured in operation 610 by approximating a value of the first physiological index corresponding to the RPE.

In operation 630, the physiological index estimating apparatus derives a personalized regression equation from the linear graph generated in operation 620.

In operation 640, the physiological index estimating apparatus estimates a second physiological index of the user at a maximal exercise level by substituting, in the personalized regression equation derived in operation 630, an RPE corresponding to the maximal exercise level.

In operation 650, the physiological index estimating apparatus evaluates an exercise capability of the user based on the second physiological index of the user estimated in operation 640. For example, the physiological index estimating apparatus may evaluate the exercise capability of the user by comparing the estimated second physiological index, for example, a VO₂ₘₐₓ and an HRₘₐₓ, to a preset standard VO₂ₘₐₓ and an HRₘₐₓ for the gender and the age of the user. Also, the physiological index estimating apparatus may estimate a plurality of second physiological indices and evaluate the exercise capability of the user based on the plurality of second physiological indices of the user.

The physiological index estimating apparatus may provide, as feedback to the user, a result of comparing the exercise capability of the user evaluated in operation 650 to a preset standard exercise capability for the gender and the age of the user.

The physiological index estimating apparatus may calculate a metabolic syndrome risk of the user based on the second physiological index estimated in operation 640, and provide a result of the calculating as feedback to the user. For example, the physiological index estimating apparatus may determine whether the second physiological index estimated in operation 640, for example, the VO₂ₘₐₓ and the HRₘₐₓ, is within a range of preset metabolic syndrome risk indices for the gender and the age of the user. The physiological index estimating apparatus may calculate a health score based on a result of the determining, or calculate a mortality risk (a risk of dying from a metabolic disease), and provide the calculated mortality risk or the metabolic syndrome risk as feedback to the user. The physiological index estimating apparatus may provide the user with a health care service including an exercise prescription, a nutrition prescription, and a lifestyle-related prescription based on the metabolic syndrome risk.

In operation 660, the physiological index estimating apparatus generates an exercise program suitable for the exercise capability of the user based on a result of the evaluating performed in operation 650. For example, when the exercise capability of the user is evaluated to be a 60% level compared to an exercise capability of a healthy person, the physiological index estimating apparatus generates an exercise program corresponding to an exercise level at the 60% level from an exercise level performed by the healthy person.

The physiological index estimating apparatus may generate an exercise program suitable for a particular purpose, for example, to lose weight, gain weight, increase a cardiopulmonary fitness of the user, or increase the exercise capability of the user.

In operation 670, the physiological index estimating apparatus adjusts an exercise level and intensity for the user based on the exercise program generated in operation 660.

During the exercise program, the physiological index estimating apparatus may adjust an exercise level for the user by providing, as feedback to the user, a real-time exercise result based on a history of performance of the user compared to a target exercise performance. In addition, after the exercise, the physiological index estimating apparatus may provide, as feedback to the user, an exercise result in response to the history of the performance of the entire exercise program, and modify the exercise program or adjust an exercise level or intensity for the user based on the exercise result.

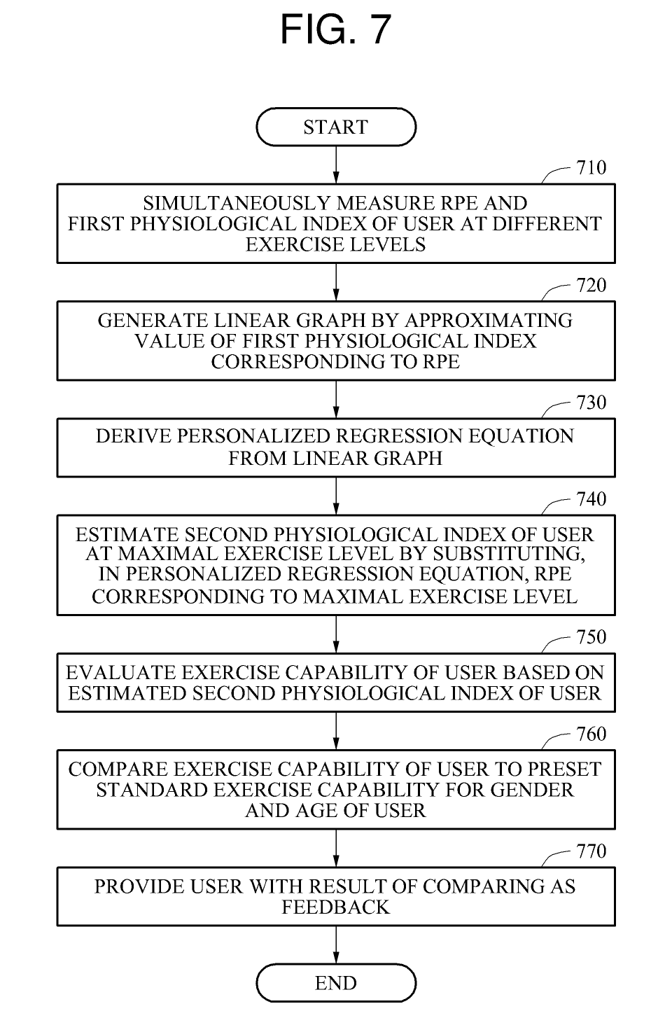

FIG. 7 is a flowchart illustrating another example of a method of estimating a physiological index of a user. For details of operations 710 through 750 in the method illustrated in FIG. 7, reference may be made to the descriptions of operations 610 through 650 in the method illustrated in FIG. 6.

In operation 760, the physiological index estimating apparatus compares the exercise capability of the user to a preset standard exercise capability for a gender and an age of the user.

In operation 770, the physiological index estimating apparatus provides a result of the comparing as feedback to the user.

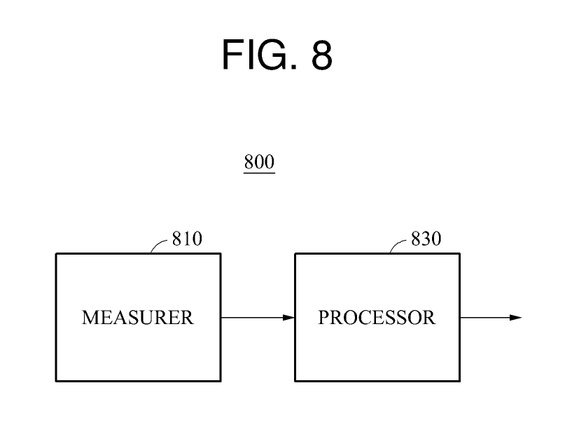

FIG. 8 is a diagram illustrating an example an example of a physiological index estimating apparatus 800.

Referring to FIG. 8, the physiological index estimating apparatus 700 includes a measurer 810 and a processor 830.

The measurer 810 measures an RPE and a first physiological index of a user at different exercise levels of an exercise having a varying exercise intensity. For example, the measurer 810 may include various sensors configured to sense the first physiological index, for example, an HR, a pulse rate, a respiratory rate, a blood pressure, a stroke volume, a cardiac output, a VE, a VO₂, a FeO₂, a FeCO₂, an EqO₂, an RER, an MET, a blood lactate level, and a SpO₂ level.

The physiological index estimating apparatus 800 inquires about an RPE of an exercise currently being performed by the user using a display (not shown) or a speaker (not shown).

The physiological index estimating apparatus 800 guides the user to select an answer in response to the inquiry from “extremely hard, hard, somewhat hard, somewhat easy, easy, and extremely easy,” or levels 0 through 10 through the display or the speaker. The physiological index estimating apparatus 800 measures the RPE of the user based on the answer or the selected level input from the user in accordance with the guidance.

The measurer 810 may be as part of the physiological index estimating apparatus 800, or may be a separate device externally located from the physiological index estimating apparatus 800. When the measurer 810 is a separate device externally located from the physiological index estimating apparatus 800, the physiological index estimating apparatus 800 receives the RPE and the first physiological index measured by the measurer 810 through a receiver (not shown).

The processor 830 estimates a second physiological index of the user at a maximal exercise level based on the RPE and the first physiological index measured by the measurer 810.

The processor 830 derives a personalized regression equation for the user based on a relationship between the RPE and the first physiological index, and estimates the second physiological index at the maximal exercise level using the personalized regression equation.

The processor 830 generates a linear or nonlinear graph by approximating a value of the first physiological index corresponding to the RPE, and derives the personalized regression equation from the generated graph. The processor 830 estimates the second physiological index at the maximal exercise level by substituting, in the personalized regression equation, an RPE corresponding to the maximal exercise level.

The processor 830 evaluates an exercise capability of the user based on the estimated second physiological index, generates an exercise program suitable for the exercise capability of the user, and adjusts an exercise level or intensity for the user based on the exercise program.

The processor 830 evaluates the exercise capability of the user based on the estimated second physiological index, compares the exercise capability of the user to a preset standard exercise capability for the gender and the age of the user, and provides a result of the comparing as feedback to the user.

The descriptions provided with reference to FIGS. 1 through 7 are also applicable to the physiological index estimating apparatus 800 illustrated in FIG. 8.

The measurer 810 and the processor 830 in FIG. 8 that perform the operations described herein with respect to FIGS. 1-8 are implemented by hardware components. Examples of hardware components include controllers, sensors, generators, drivers, memories, comparators, arithmetic logic units, adders, subtractors, multipliers, dividers, integrators, and any other electronic components known to one of ordinary skill in the art. In one example, the hardware components are implemented by computing hardware, for example, by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein with respect to FIGS. 1-7. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1, 2, 6, and 7 that perform the operations described herein with respect to FIGS. 1-8 are performed by a processor or a computer as described above executing instructions or software to perform the operations described herein.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of estimating a physiological index of a user, the method comprising:
measuring a rating of perceived exertion (RPE) and a first physiological index of a user at different exercise levels of an exercise or a daily activity having a varying exercise intensity; and
estimating a second physiological index of the user at a maximal exercise level based on the RPE and the first physiological index.

2. The method of claim 1, wherein the estimating of the second physiological index comprises:
deriving a personalized regression equation for the user based on a relationship between the RPE and the first physiological index; and
estimating the second physiological index at the maximal exercise level using the personalized regression equation.

3. The method of claim 2, wherein the deriving of the personalized regression equation comprises:
generating a graph based on the relationship between the RPE and the first physiological index; and
deriving the personalized regression equation from the graph.

4. The method of claim 3, wherein the generating of the graph comprises generating the graph by approximating a value of the first physiological index corresponding to the RPE.

5. The method of claim 2, wherein the estimating of the second physiological index comprises estimating the second physiological index at the maximal exercise level by substituting, in the personalized regression equation, an RPE corresponding to the maximal exercise level.

6. The method of claim 1, wherein the RPE and the first physiological index are simultaneously measured at the different exercise levels.

7. The method of claim 1, wherein the RPE is expressed by a Borg scale, an OMNI scale, a Likert scale, or a visual analog scale of perceived exertion.

8. The method of claim 1, wherein the first physiological index comprises any one or more of a heart rate, a pulse rate, a respiratory rate, a blood pressure, a stroke volume, a cardiac output, a ventilation (VE), an oxygen uptake (VO₂), an oxygen concentration in exhaled air (FeO₂), a carbon dioxide concentration in exhaled air (FeCO₂), a ventilatory equivalent for oxygen (EqO₂), a respiratory exchange ratio (RER), a metabolic equivalent of task (MET), a blood lactate level, and a blood oxygen saturation (SpO₂) level.

9. The method of claim 1, wherein the different exercise levels comprise at least two exercise levels selected from exercise levels corresponding to a warming-up stage prior to an exercise, an exercising stage during the exercise, and a cooling-down stage subsequent to the exercise.

10. The method of claim 1, further comprising evaluating an exercise capability of the user based on the estimated second physiological index.

11. The method of claim 10, further comprising:
generating an exercise program suitable for the exercise capability of the user based on a result of the evaluating; and
adjusting an exercise level and intensity for the user based on the exercise program.

12. The method of claim 10, further comprising:
comparing the exercise capability of the user to a preset standard exercise capability for a gender and an age of the user; and
providing a result of the comparing as feedback to the user.

13. The method of claim 1, further comprising calculating a metabolic syndrome risk of the user based on the estimated second physiological index.

14. A non-transitory computer-readable storage medium storing instructions to control computing hardware to perform the method of claim 1.

15. An apparatus for estimating a physiological index of a user, the apparatus comprising:
a measurer configured to measure a rating of perceived exertion (RPE) and a first physiological index of a user at different exercise levels of an exercise having a varying exercise intensity; and
a processor configured to estimate a second physiological index of the user at a maximal exercise level based on the RPE and the first physiological index.

16. The apparatus of claim 15, wherein the processor is further configured to derive a personalized regression equation for the user based on a relationship between the RPE and the first physiological index, and estimate the second physiological index at the maximal exercise level using the personalized regression equation.

17. The apparatus of claim 16, wherein the processor is further configured to generate a graph by approximating a value of the first physiological index corresponding to the RPE, and derive the personalized regression equation from the graph.

18. The apparatus of claim 16, wherein the processor is further configured to estimate the second physiological index at the maximal exercise level by substituting, in the personalized regression equation, an RPE corresponding to the maximal exercise level.

19. The apparatus of claim 15, wherein the processor is further configured to evaluate an exercise capability of the user based on the estimated second physiological index, generate an exercise program suitable for the exercise capability of the user, and adjust an exercise level and intensity for the user based on the exercise program.

20. The apparatus of claim 15, wherein the processor is further configured to evaluate the exercise capability of the user based on the estimated second physiological index, compare the exercise capability of the user to a preset standard exercise capability for a gender and an age of the user, and provide a result of the comparing as feedback to the user.
